(19) United States
(12) Patent Application Publication
(10) Pub. No.: US 2007/0173831 A1
(43) Pub. Date: Jul. 26, 2007

(54) DEVICE AND METHOD FOR THE PLACEMENT OF SPINAL FIXATORS

(76) Inventor: M. S. Abdou, San Diego, CA (US)

Correspondence Address: FISH & RICHARDSON, PC, P.O. BOX 1022, MINNEAPOLIS, MN, 55440-1022, US

(21) Appl. No.: 11/559,871

(22) Filed: Nov. 14, 2006

Related U.S. Application Data
(60) Provisional application No. 60/736,387, filed on Nov. 14, 2005.

(51) Int. Cl. A61F 2/30

(52) U.S. Cl. 606/61

(57) ABSTRACT

An instrument is adapted for placing a cross-member between a pair of bone screws. The instrument includes an actuator and a housing that contains the cross-member in a pre-deployed state such that the cross-member is at least partially enclosed within the housing. A mechanism couples the actuator to the cross-member. Actuation of the actuator causes the mechanism to rotate the cross-member out of the housing and to further translate the cross-member in a direction toward engagement with a bone screw.

### REFERENCE TO PRIORITY DOCUMENT

This application claims priority of co-pending U.S. Provisional Patent Application Ser. No. 60/736,387, filed Nov. 14, 2005. Priority of the aforementioned filing date is hereby claimed and the disclosure of the Provisional Patent Application is hereby incorporated by reference in its entirety.

### BACKGROUND

The present disclosure is directed at systems and methods that stabilize the bony skeleton, components thereof, and methods of implant placement. These systems can be used to adjust, align and maintain the spatial relationship(s) of adjacent bones or bony fragments during post-operative healing.

In spine surgery, the operative field is situated deep within the body cavities and exposure of the spine often requires significant dissection of normal surrounding tissues. The unnecessary disruption of neighboring tissues can lead to a significant increase in scar formation, the risk of infection, peri-operative pain and an increase in the time needed for post-operative recovery. Since these operations are frequently done to relieve the pain caused by spinal pathology, extensive dissection of the tissues around the spine will actually decrease the overall pain relieving effect of the procedure. For these reasons, there has been tremendous interest in devising methods for spinal stabilization with minimal disruption of neighboring tissues.

Screw placement into the vertebral bodies has become a common method for attachment of spinal fixation devices. This approach is particularly common in fixation of the thoracic and lumbar spine. However, the surgical exposure of these spinal segments requires extensive dissection of the para-spinal musculature and other important neighboring tissues. The negative physiologic effects of tissue dissection and injury have fueled the search for less invasive ways to place these devices.

Percutaneous placement of screws or other fasteners into the spinal vertebral bodies can be readily accomplished. Since these screws are often placed into the spine in the horizontal plane and perpendicular to the long axis of the spine (vertical plane), the spine can be radiographically visualized and the screws placed through a small skin incision that is slightly larger than the diameter of the largest segment of the screw assembly. However, the cross-member that connect the individual screws must be placed along the long axis of the spine, making the minimally invasive placement of these connectors much more difficult.

### SUMMARY

Disclosed are systems and methods for minimally invasive placement of spinal fixation devices. Bone fasteners are placed into the pedicles of the vertebral bodies of the spinal segments to be stabilized. The fasteners are placed through small skin incisions that are sufficiently large to accommodate the diameter of the fastener and fastener placement device. The location of the bony elements within the body cavity is identified using radiography, stereotactic guidance and/or the like. Each fastener is driven into the desired vertebral bodies with one end of a hollow cylindrical member attached to it. The other end of the cylindrical member resides above the skin surface and outside the body. The cylindrical member provides an access route to the proximal end of each bone fastener.

A frame is attached to the external and free end of the cylindrical members. The external frame positions and fixates the cylindrical members in a co-planar configuration and it also permits the measurement of the distance between them. The stabilization cross-member that interconnects the bone fasteners is implanted using a placement device. That device is advanced into the hollow center of a cylindrical member attached to one bone fastener and used to rotate the cross-member into position between the free ends of one or more of the remaining fasteners. Once in place, the cross-member is held stationary by the locking mechanism of the one or more remaining bone fasteners while the placement device from the cross-member and removed.

Several embodiments of the cross-member placement device are illustrated. In one embodiment, a hand-actuated device is disclosed wherein closure of a handle causes an attached cross-member to rotate, translate and be positioned into a pre-determined location relative to the distal end of the placement device. Another embodiment of the device with a different internal mechanism is also shown. In other embodiments, the device is actuated by the rotation of a member on one end of the device and that member is adapted to be rotated by a hand or power activated drill. A knife-like adapter is also illustrated. This adapter can be attached onto the distal end of any of the illustrated cross-member placement devices and then used to divide the tissues between the cylindrical fastener holding members before the cross-member is implanted.

In one aspect, there is disclosed an instrument for placing a cross-member between a pair of bone screws, comprising; an actuator; a housing coupled to the actuator, the housing containing the cross-member in a pre-deployed state wherein the cross-member is at least partially enclosed within the housing; and mechanism coupling the actuator to the cross-member, wherein actuation of the actuator causes the mechanism to rotate the cross-member out of the housing and to further translate the cross-member in a direction toward engagement with a bone screw.

In another aspect, there is disclosed a system for placing a cross-member between first and second bone screws, comprising: an instrument comprising: (a) an actuator; (b) a housing coupled to the actuator, the housing containing the cross-member in a pre-deployed state wherein the cross-member is at least partially enclosed within the housing; and (c) a mechanism coupling the actuator to the cross-member, wherein actuation of the actuator causes the mechanism to rotate the cross-member out of the housing and to further translate the cross-member in a direction toward engagement with a bone screw. The system further comprises a mounting system including at least one mounting member that aligns the instrument within a plane that includes the first and second bone screws.

In another aspect, there is disclosed an instrument for positioning a device relative to a pair of bone screws, comprising: an actuator; a housing coupled to the actuator, the housing containing the device in a pre-deployed state wherein the device is at least partially enclosed within the housing; and a mechanism coupling the actuator to the device, wherein actuation of the actuator causes the mechanism to rotate the device out of the housing and to further translate the device in a direction toward the bone screws.

In another aspect, there is disclosed an instrument for positioning a device relative to a pair of bone screws, comprising: an actuator; a housing coupled to the actuator, the housing containing the device in a pre-deployed state wherein the device is at least partially enclosed within the housing; and an mechanism coupling the actuator to the device, wherein actuation of the actuator causes the mechanism to rotate the device out of the housing in a direction toward the bone screws.

In another aspect there is disclosed an instrument for positioning a device relative to a pair of bone screws, comprising: an actuator; a housing coupled to the actuator, the housing containing the device in a pre-deployed state wherein the device is at least partially enclosed within the housing; and a mechanism coupling the actuator to the device, wherein actuation of the actuator causes the mechanism to translate the device out of the housing in a direction toward the bone screws.

In another aspect, there is disclosed a method of placing an implant relative to a pair of bone screws, comprising: providing an instrument having the implant contained at least partially within the instrument actuating the instrument so that the instrument causes the implant to rotate out of the instrument and translate toward a deployment position relative to the bone screws; and attaching the implant to the bone screws.

The placement instrument, system and method described herein provide ease of use as well as reliable and intuitive rod delivery through a minimally invasive approach. Other features and advantages should be apparent from the following description of various embodiments, which illustrate, by way of example, the principles of the disclosed devices and methods.

### BRIEF DESCRIPTION OF THE DRAWINGS

FIG. 1 shows a perspective view of an instrument that is adapted to install an implant onto the spine.

FIG. 2 shows the instrument with an implant in a deployed state between a pair of bone fasteners.

FIGS. 3 and 4 show perspective views of the instrument.

FIG. 5 shows an enlarged view of a distal region of the instrument housing.

FIG. 6 shows an exploded view of the instrument.

FIGS. 7 and 8 show enlarged, exploded views of a distal region of the instrument.

FIGS. 9 and 10 show the mechanical interaction between an actuation handle and internal deployment mechanism off the instrument.

FIG. 11 shows an enlarged view of the distant region of the instrument housing in an assembled state.

FIG. 12 shows the instrument after actuation such that the cross-member has rotated and translated to a deployment position.

FIG. 13A shows an enlarged view of the distal region of the instrument with the cross-member in the deployment state.

FIGS. 13B and 13C show cross-sectional views of the cross-member attachment holder with a retainer engaging and disengaging the cross-member.

FIG. 14 shows a bone screw prior to insertion into a vertebral body V1 and a bone screw already inserted into a vertebral body V2.

FIG. 15 shows support members of a platform system attached to the bone screws.

FIG. 16 shows the platforms of the mounting system prior to attachment to the support members.

FIG. 17 shows measurement hatch marks of the instrument mounting system.

FIG. 18 shows a perspective view of the assembled mounting system attached to the vertebral bodies V1 and V2.

FIG. 19 shows the instrument fully seated in the mounting system.

FIG. 20 shows another embodiment of a device that can be used with the instrument.

FIG. 21 shows the instrument with the knife member positioned in a pre-deployed state.

FIG. 22 shows the instrument of FIG. 21 in an exploded state.

FIGS. 23 and 24 show enlarged, exploded views of the distal region of the instrument.

FIG. 25 shows another embodiment of the instrument that includes an alternate embodiment of the internal actuation mechanism.

FIG. 26 shows an exploded view of the instrument of FIG. 25.

FIG. 27 shows an enlarged view of an exemplary actuation mechanism.

FIG. 28 shows the distal region of the instrument with a portion of the mechanism removed.

FIGS. 29 and 30 snow the action of the mechanism during actuation of the instrument.

FIGS. 31A and 31B show top and side views of an instrument that is actuated by coupling to a hand-powered or motorized drill.

FIG. 32 shows an exemplary embodiment of an internal actuation mechanism.

### DETAILED DESCRIPTION

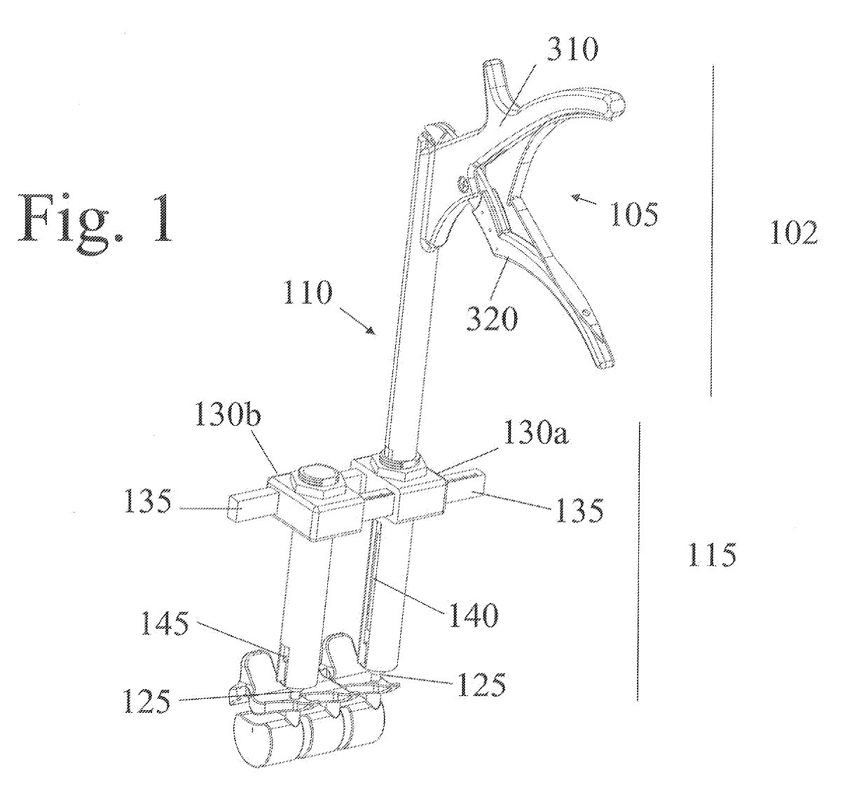

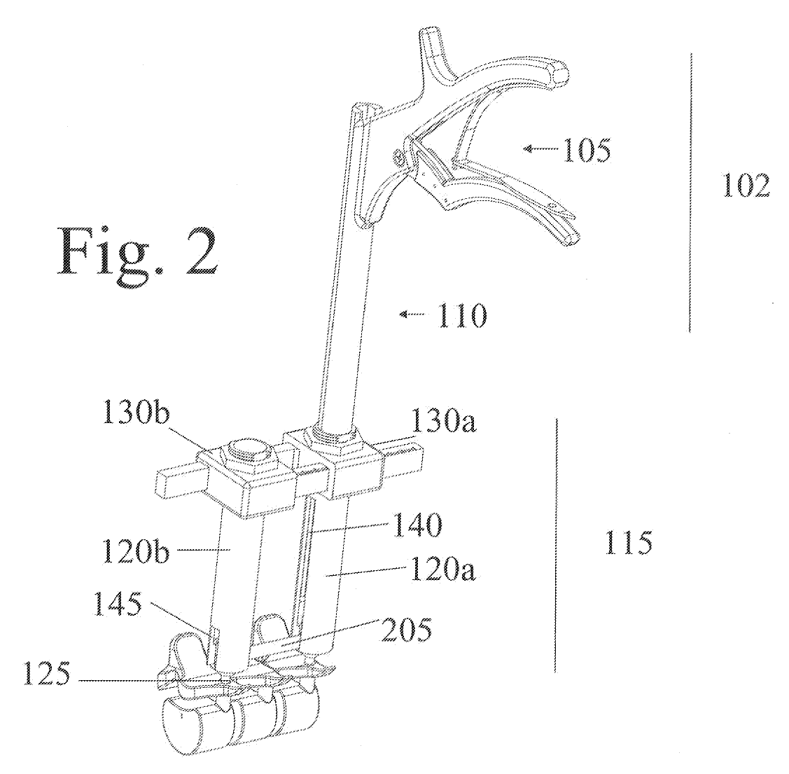

FIG. 1 shows a perspective view of an instrument 102 that is adapted to install an implant onto the spine. The implant can be, for example, a cross-member, such as an interconnecting rod or it can be a different type of implant. FIG. 2 shows the instrument 102 with an implant comprised of a cross-member 205 in a deployed state between a pair of bone fasteners. The instrument 102 includes an actuator 105 for actuating the instrument and a housing 110 that contains the cross-member 205 (FIG. 2) prior to installation. The instrument 102 is actuated to cause the cross-member to deploy out of the housing 110 and into an installed position such as by actuation of an internal mechanism. In one embodiment, the instrument deploys the cross-member by causing the cross-member to rotate out of the housing 110 and translate (i.e., move without rotating) toward a deployment location, as described in greater detail below. When deployed, the cross-member 205 links a pair of bone fasteners 125, as shown in FIG. 2.

With reference still to FIGS. 1 and 2, the instrument 102 is configured to couple to a mounting system 115 that supports the instrument 102 in predetermined orientation during installation of the cross-member 205. The mounting system 115 includes a pair of support members 120a and 120b that removably couple to bone fasteners 125 attached to underlying vertebral bodies. The mounting system 115 further includes a pair of platforms 130a and 130b that are positioned atop each of the support members 120a and 120b, respectively The support member 120a/platform 130a are slidably attached to the support member 120b/platform 130b via one or more external cross-members 135 that can function as means for measuring the cross-member 205, as described in detail below.

The support, members 120 are elongated structures that extend outwardly from the bone fasteners 125. In the illustrated embodiment, the support members 120 are elongated, cylindrical tubes although the shape of the support members 120 can vary. Each support member 120 has an internal shaft that sized to receive at least a portion of the instrument 102, such as the housing 110. Each support member 120 has an elongated slot 140 that extends from a distal end of the support member to a proximal region of the support member. In addition, each support member 120 has a second elongate slot 145 located at a, 180 degree angle relative to the first elongated slot 140. The second elongated slot 145 can have a length that is shorter than the first elongated slot 140.

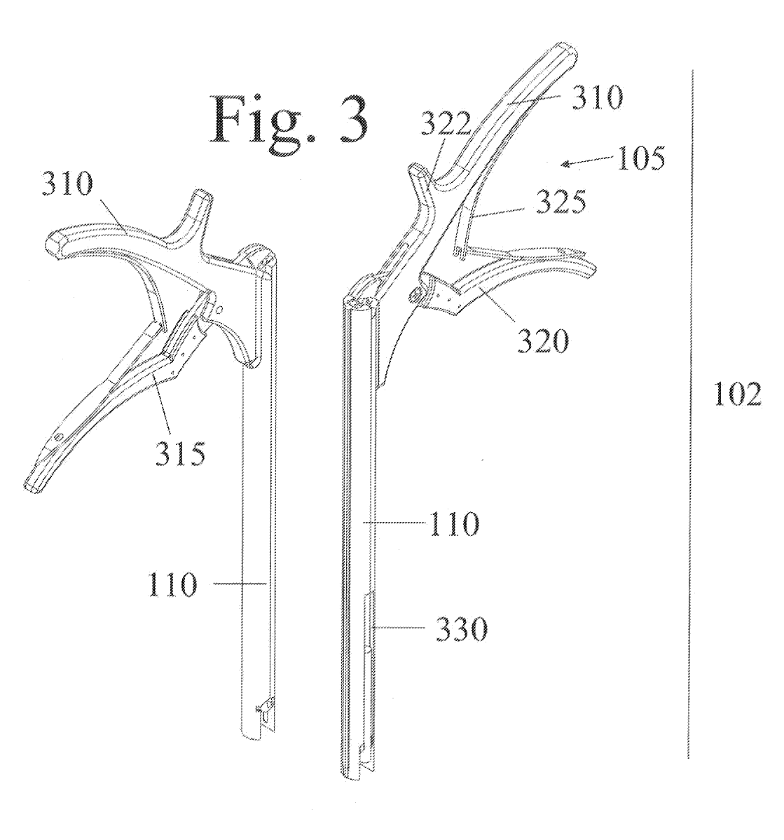

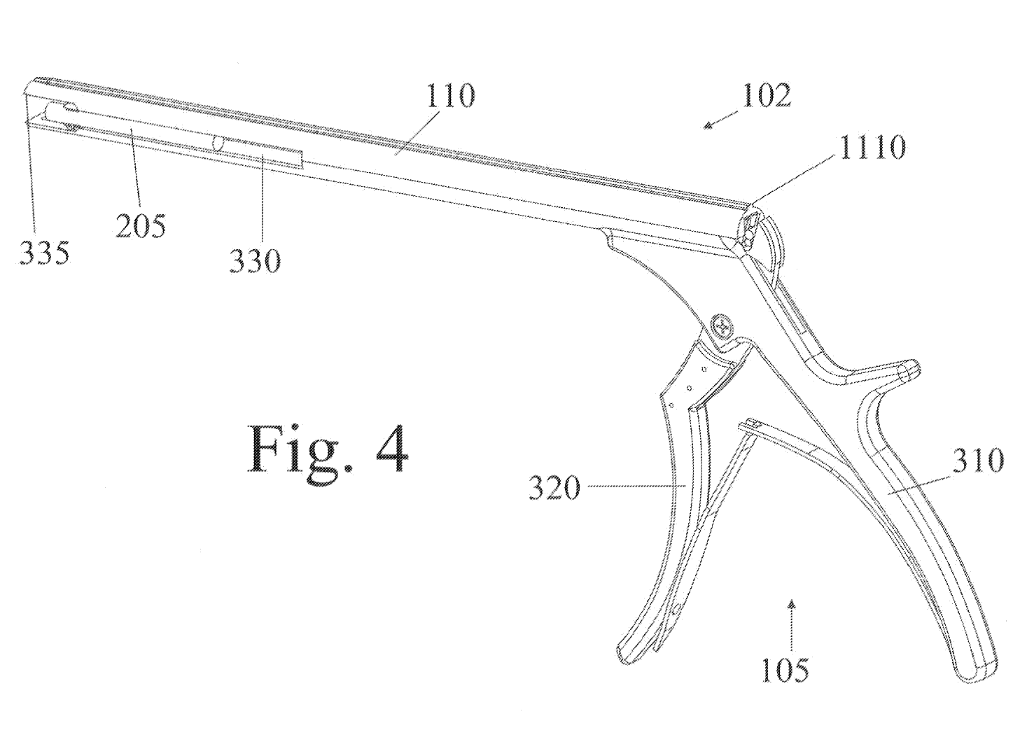

FIGS. 3 and 4 show perspective views of the instrument 102. In the illustrated embodiment, the actuator 105 is a two-piece handle having a first arm 310 and a second arm 320 that is movably mounted relative to the first arm 310 in a pivot or trigger fashion. The first and second arms are ergonomically arranged such that an operator can grasp the arms using a single hand. For example, the first arm 310 is sized and shaped to support an operator's palm and thumb such as on a thumb grip 322. Likewise, the second arm 320 can be grasped by the operator's fingers to pull the second arm 320 toward the first arm 310 and actuate the instrument 102. A biasing member 325 is interposed between the first and second arms. It should be appreciated that the actuator 105 can take on other forms and mechanisms and need not be in a two-piece handle configuration.

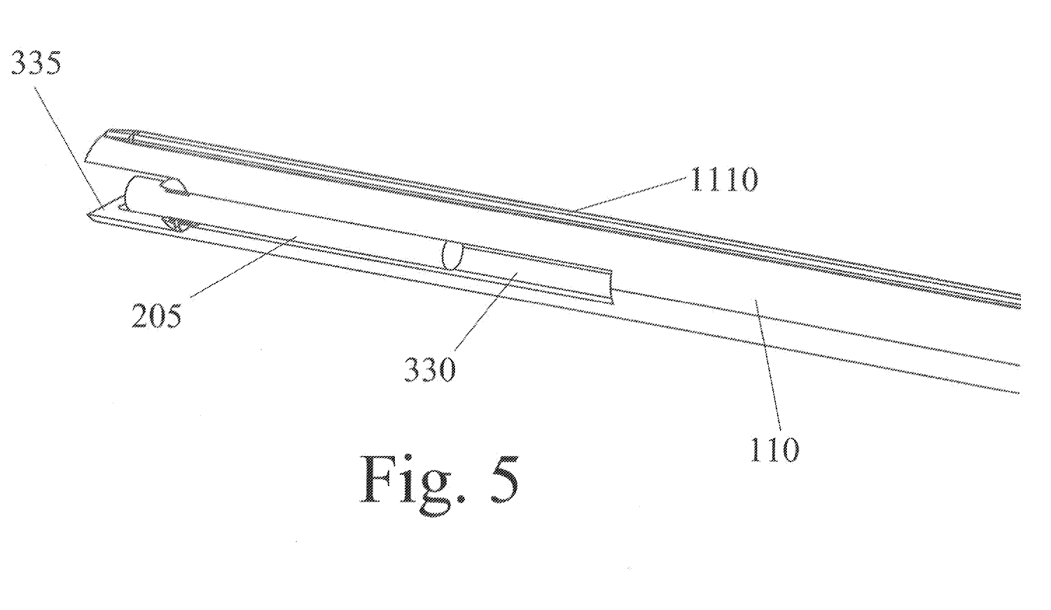

With reference still to FIGS. 3 and 4, the housing 110 extends outward from the handle 105. The housing 110 is sized and shaped to contain the cross-member 205 (FIG. 4). In the illustrated embodiment, the housing 110 has an elongated tube-like shape and is partially hollow so as to contain the cross-member 205 as well as an internal actuation mechanism that couples the actuator to the cross-member, as described in detail below. FIG. 5 shows an enlarged view of the distal region of the instrument housing 110. A slot 330 is located at or near a distal end 335 of the housing 110. The slot 330 communicates with an internal cavity in which the cross-member 205 resides. The slot 330 is sufficiently long and wide such that the cross-member 205 can pass through the slot 330 during deployment of the cross-member 205. The distal end 335 of the housing 110 includes a distal opening that is sufficiently sized to permit passage of cross-member 205 and its holder.

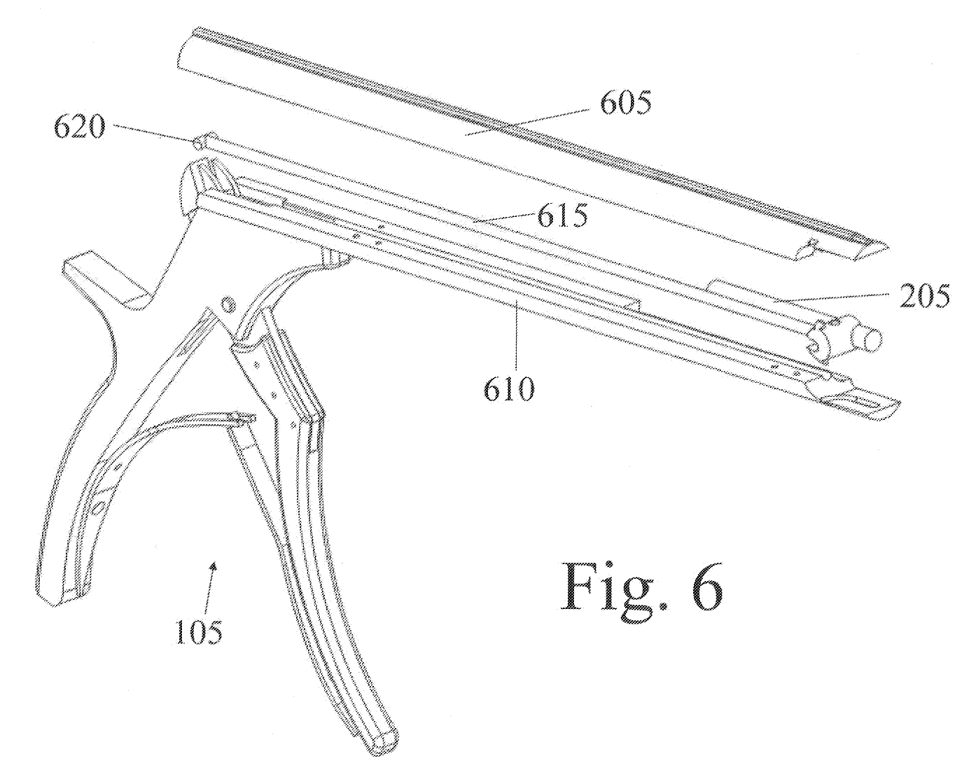

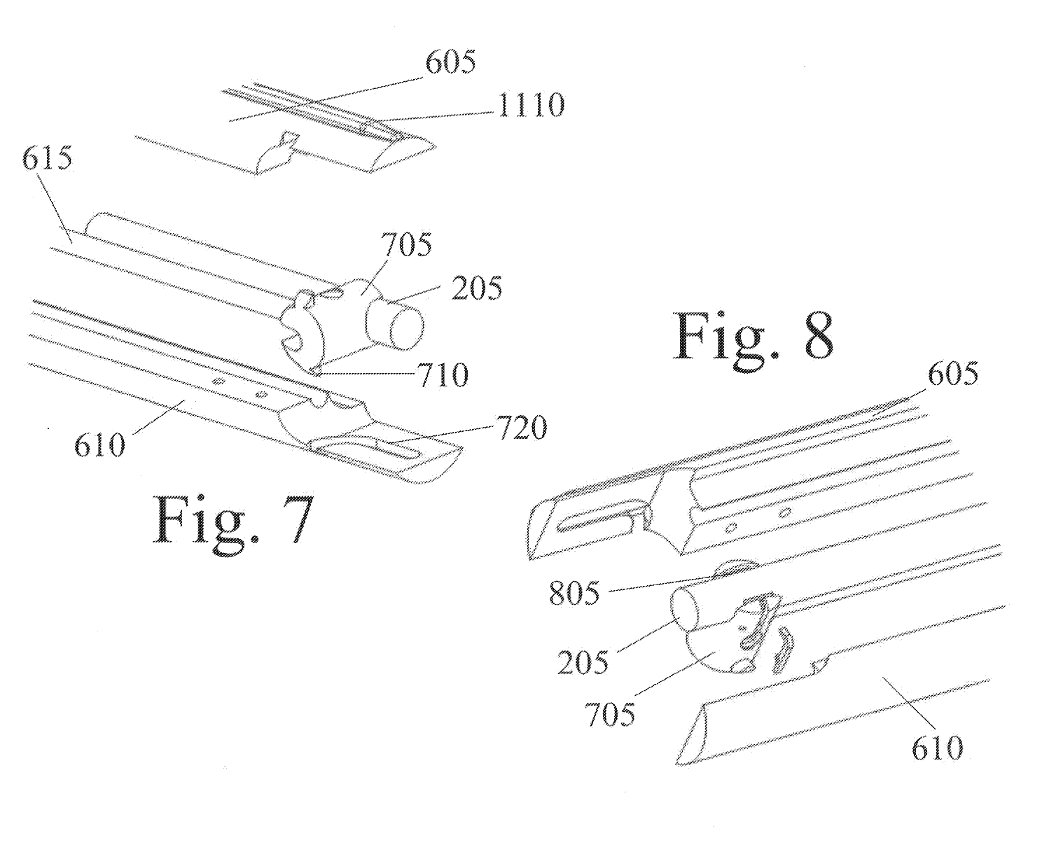

FIG. 6 shows an exploded view of the instrument 102 whiles FIGS. 7 and 8 show enlarged, exploded views of a distal region of the instrument. With reference to FIGS. 6-8, the housing 110 is formed of an upper member 605 and a lower member 610 that collectively define an internal region. A deployment member 615 is positioned within the internal region of the housing. The deployment member 615 interfaces with and deploys the cross-member 205 upon actuation of the instrument. The deployment member 615 can vary in shape and in mechanism. In the illustrated embodiment, the deployment member 615 is an elongate rod having a proximal end 620 (FIG. 6) coupled to the actuation handle 105 such as via an attachment to the second arm 320.

With reference to FIGS. 7 and 8, a cross-member attachment holder 705 is mounted on a distal end of the deployment member 615. The cross-member attachment holder 705 removably holds the cross-member 205 in a pre-deployed state inside the housing. In this regard, the cross-member holder 705 includes a slot 805 (FIG. 8) in which the cross-member 205 is positioned. The cross-member attachment holder 705 has a projection 710 that slidably mates with a pre-shaped guideway 720 (FIG. 7). During actuation of the instrument 102, the actuator interacts with the deployment member 615, which then moves the cross-member attachment holder 705 along the guideway 720 to cause the cross-member 205 to rotate and translate to a deployment position.

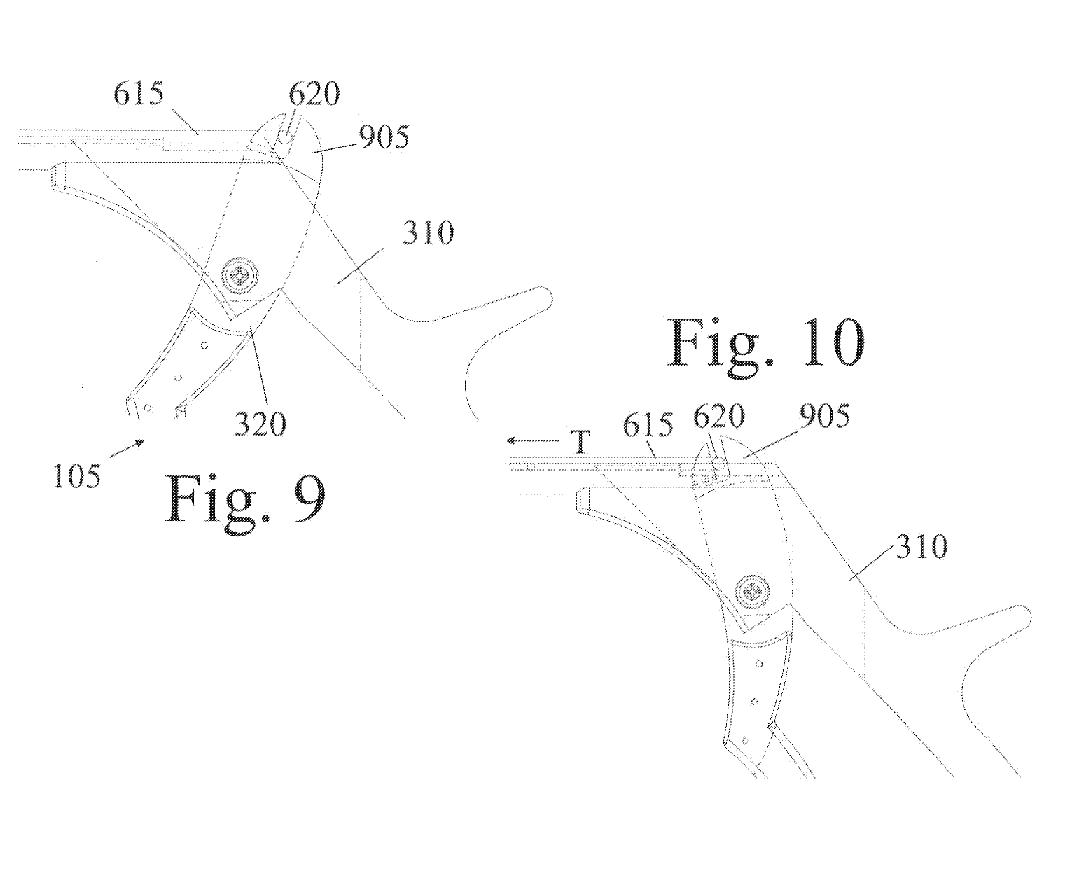

This is described in more detail with reference to FIGS. 9 and 10, which show the mechanical interaction between the actuation handle 105 and the internal deployment mechanism of the instrument 102. The second arm 320 of the handle has an upper end 905 that is attached to the proximal end 620 of the elongated deployment member 615. FIG. 9 shows the actuation handle 105 prior to actuation wherein the second arm 320 has not been pulled toward the first arm 310. The upper end 905 of the second arm 320 is at a first position. When the instrument is actuated (such as by pulling the second arm 320 toward the first arm 310 as shown in FIG. 10), the upper end 905 of the second arm moves forward and pushes the deployment member 615 towards distal end 335, as represented by the arrow T in FIG. 10.

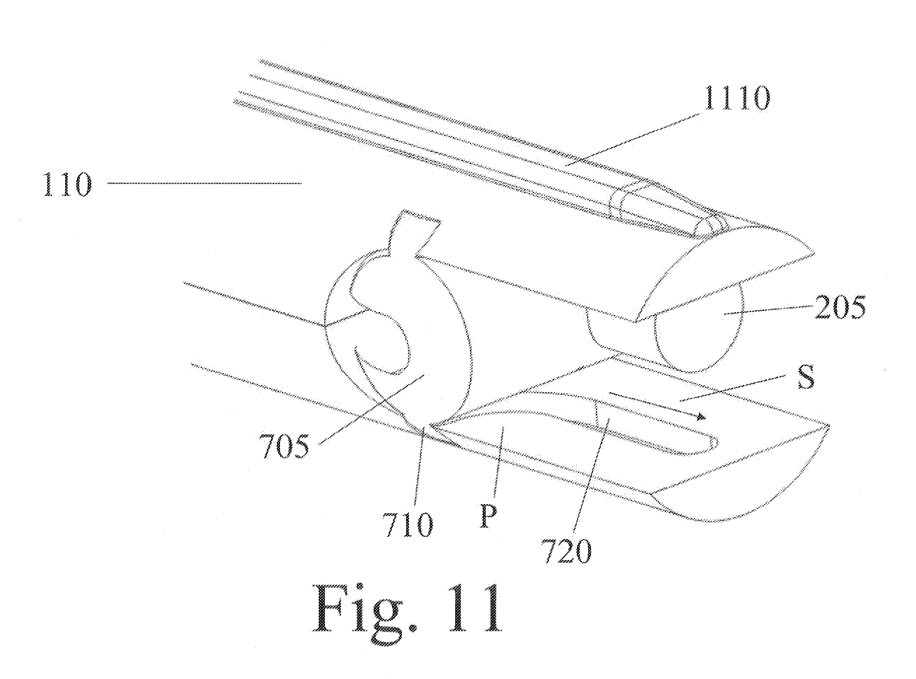

As the deployment member 615 moves towards the distal end of the device, the cross-member attachment holder 705 (FIG. 7) is forcefully rotated and translated because of the interaction between projection 710 and guideway 720. This is explained in more detail with reference to FIG. 11, which shows an enlarged view of the distal region of the instrument housing 110 in an assembled state. As mentioned, the projection 710 of the cross-member attachment holder 705 is slidably positioned within the guideway 720. The guideway 720 is shaped such that, upon actuation of the instrument 102, the cross-member attachment holder 705 moves along the guideway 720 in a manner that rotates and/or translates the cross-member 205. The guideway 720 is shaped such that the cross-member attachment holder 705 pivots or rotates about a pivot point P as the cross-member attachment holder 705 reaches the curved point along the guideway 720. This causes the attached cross member 205 to pivot outward such that it is oriented transverse or cross-wise to the axis of the housing 110. The cross-member attachment holder 705 then translates in the direction S as it travels along the straight section of the guideway 720 to thereby move the cross-member 205 in the distal direction. In an alternate embodiment, the cross-member rotates but does not translate. In another embodiment, the cross-member translates but does not rotate.

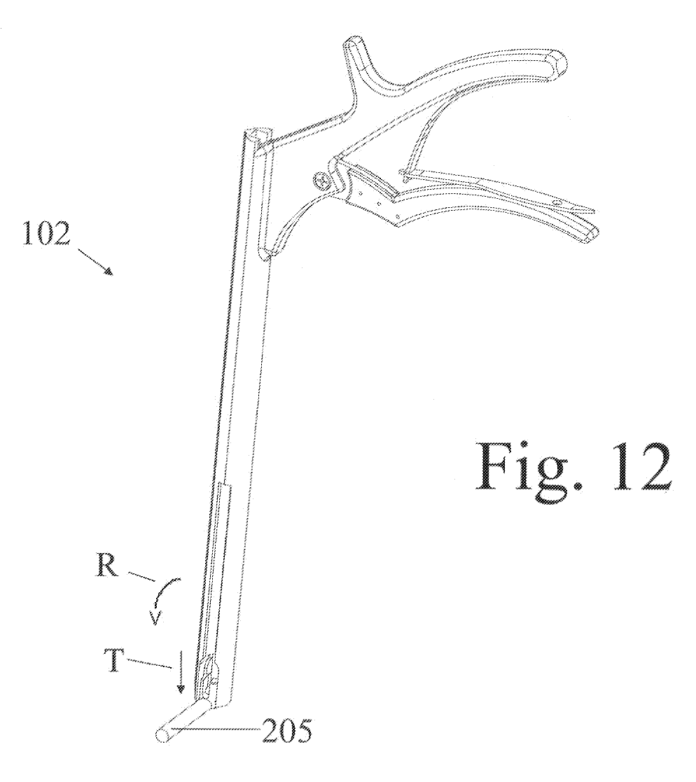

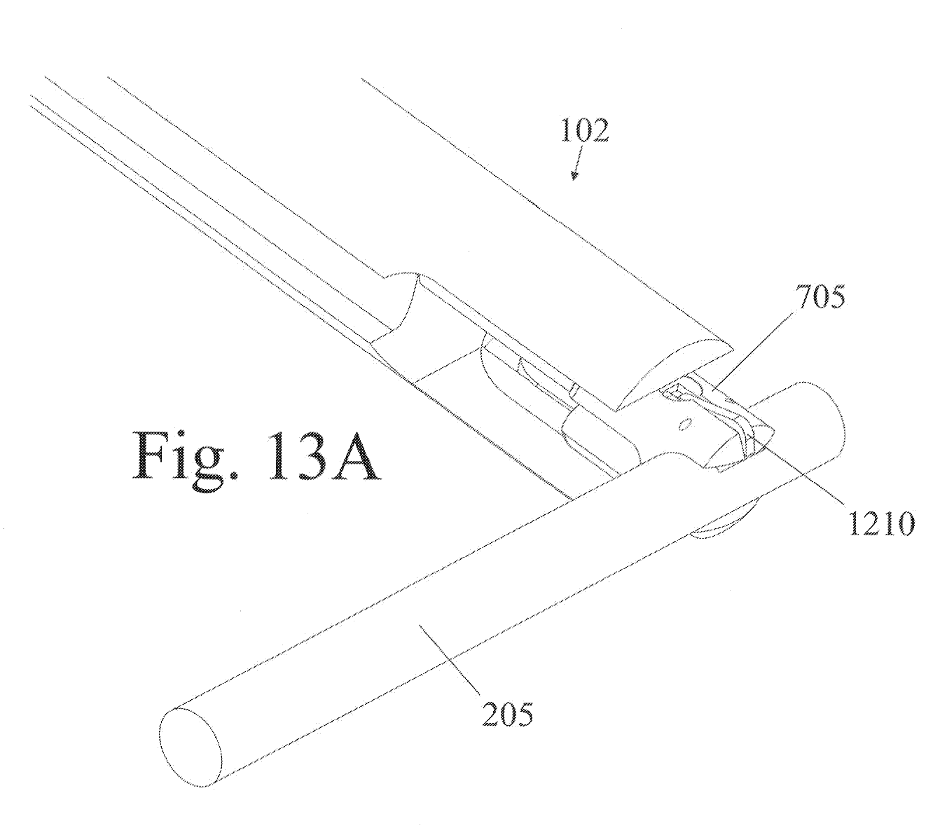

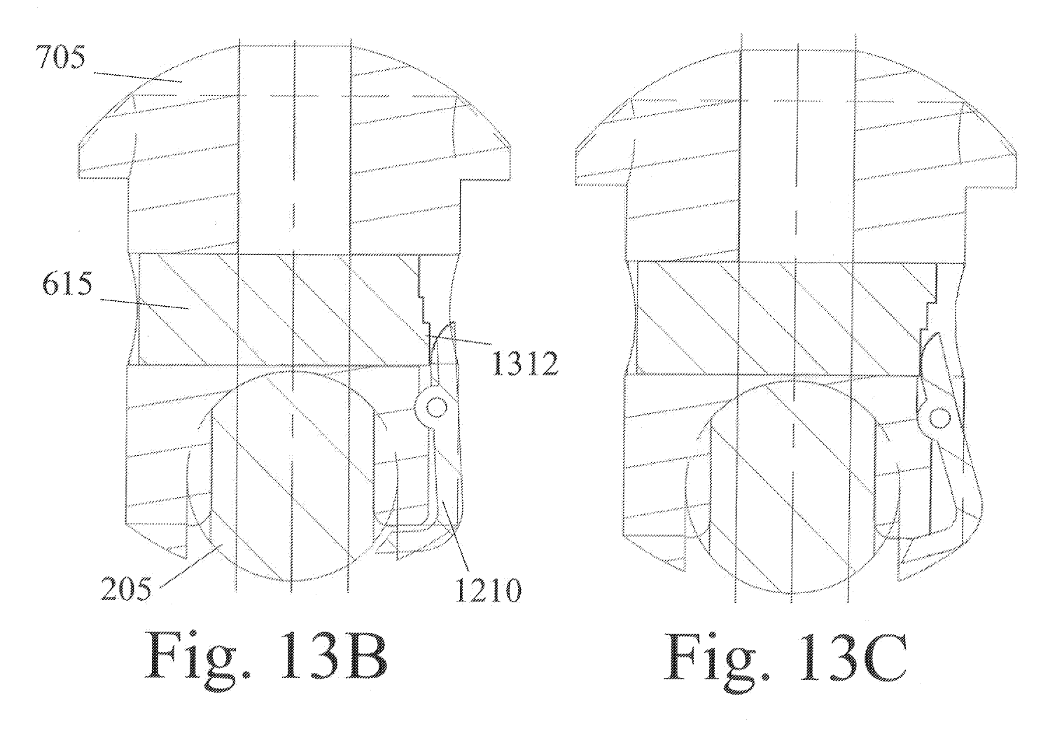

FIG. 12 shows the instrument 102 after actuation such that the cross-member 205 has rotated (as represented by the arrow R) and translated (as represented by the arrow T) to a deployment position. For examples in the deployment position, the cross-member 205 may be in an orientation that is transverse to the axis of the housing 110 as shown in FIG. 12, or it can be in some other predetermined orientation. FIG. 13A shows an enlarged view of the distal region of the instrument 102 with the cross-member 205 in the deployment state. The cross-member attachment holder 705 includes a retainer 1210, such as a clip, that removably engages the cross-member 205 to retain the cross-member 205 within the cross-member attachment holder 705. The retainer 1210 is designed to automatically retain member 205 within attachment holder 705 when the device is in the un-deployed positioned FIG. 4) and to automatically release member 205 when the device is in the deployed position (FIG. 12). While the cross-member attachment retainer mechanism is detailed further below, it should be appreciated that other retainer mechanisms may be alternatively employed.

FIGS. 13B and 13C show cross-sectional view of the cross-member attachment holder with a retainer engaging and disengaging the cross-member. In the engaged state shown in FIG. 13B, the retainer 1210 engages the cross-member 205. A T-shaped portion of the deployment member 615 is rotatably mounted within the holder 705. The T-shaped portion of the deployment member 615 has a ramped surface 1312 that abuts the retainer 1210 prior to deployment. This prevents the retainer 1210 from pivoting outward and keeps the cross-member positioned within the holder 705. As the instrument is actuated, the T-shaped portion of the deployment member 615 rotates within the holder 705 such that the ramped surface 1312 moves away from the retainer 1210, as shown in FIG. 13C. The retainer 1210 is then free to disengage from the holder 705 and the cross-member 205 can be removed.

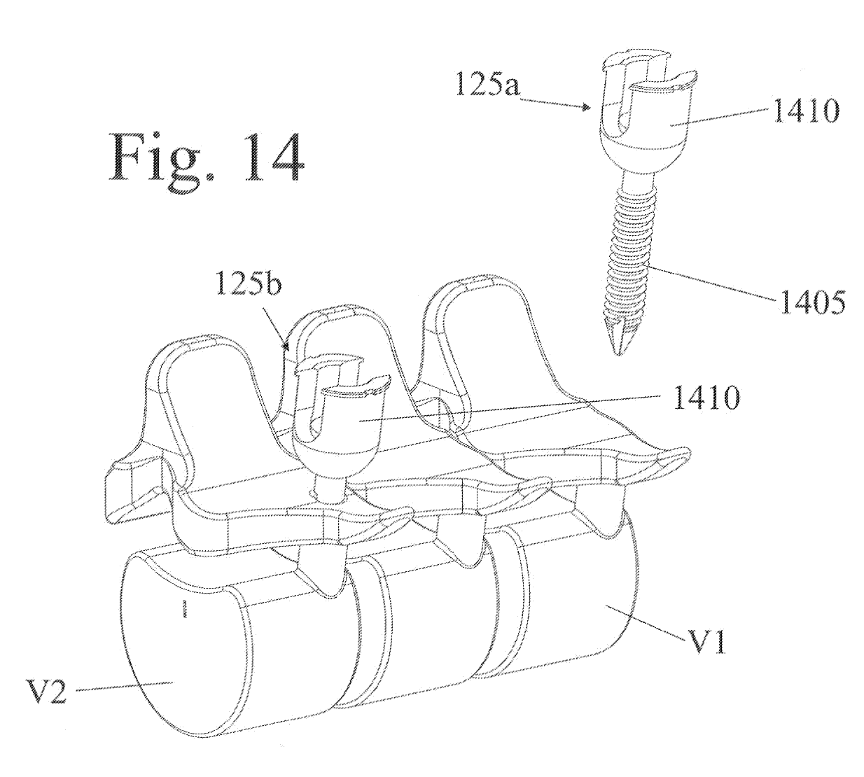

An exemplary method of using the instrument 102 is now described. In an initial step, at least one bone fastener, such as a bone screw 125 is inserted into a bone structure, such as a vertebral body. FIG. 14 shows a bone screw 125a prior to insertion into a vertebral body V1 and a bone screw 125b already inserted into a vertebral body V2. The type of bone screw can vary. In the illustrated embodiment, each bone screw 125 has an elongated shank 1405 adapted to be screwed into bone. The shank 1405 is attached to a receiver member 1410 that is adapted to receive a cross-member for linking the two bone screws. The receiver member 1410 can be coupled to the shank 1405 in a mono-axial or poly-axial configuration as will be known to those of skill in the art. In addition, the instrument 102 is not limited to use with the specific type of bone screw shown in FIG. 14.

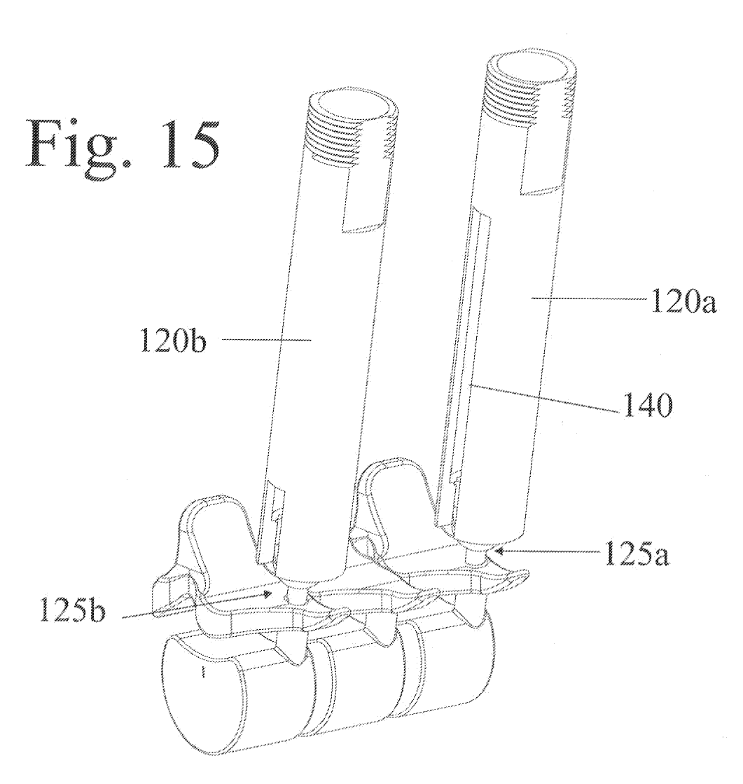

FIG. 15 shows the support members 120 of the platform system attached to the bone screws 125. In one embodiment, the screw and attached support member are already attached at the time of screw insertion into bone. In another embodiment, the screws are attached to the bone and the support members are then attached to the screws. The support members 120 each have a distal opening that is sized to fit over the receiver member (or some other portion) of the respective bone screws 125. The support members 120 extend outwardly from the bone screws 125 such that the support members 120 are oriented within the same plane and in a generally parallel configuration. In addition, the support members are oriented with the elongated slots 140 facing one another.

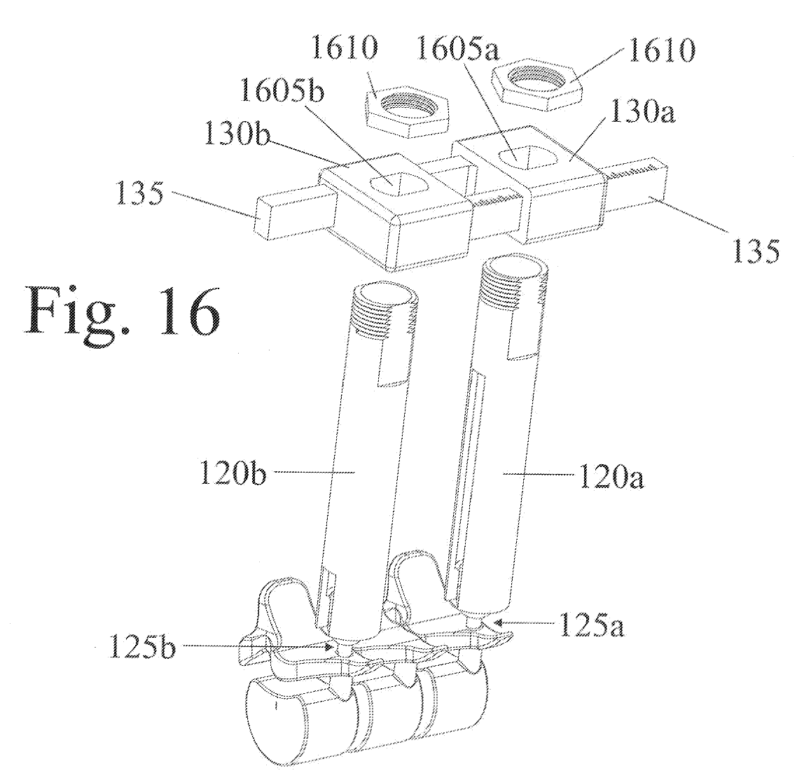

FIG. 16 shows the platforms 130 of the mounting system prior to attachment to the support members 120. Each platform 130 has a mounting hole 1605 that is sized to receive a proximal region of a respective support member 120. As mentioned, the platforms 130 are slidably movable relative to one another via at least one external cross-member 135. Prior to installing the platform 130 onto the support members 120, the distance between the platforms 130 is adjusted to correspond to the distance between the support members 120 and the attached bone screws 125. Thus, the distance is adjusted such that the apertures 1605 in each platform 130a/130b simultaneously align with corresponding support members 120a/120b. The platforms 130 can then be inserted onto the support members 120 and secured thereto, such as by using locking nuts 1610 that mate with threaded surfaces on the proximal ends of the support members 120.

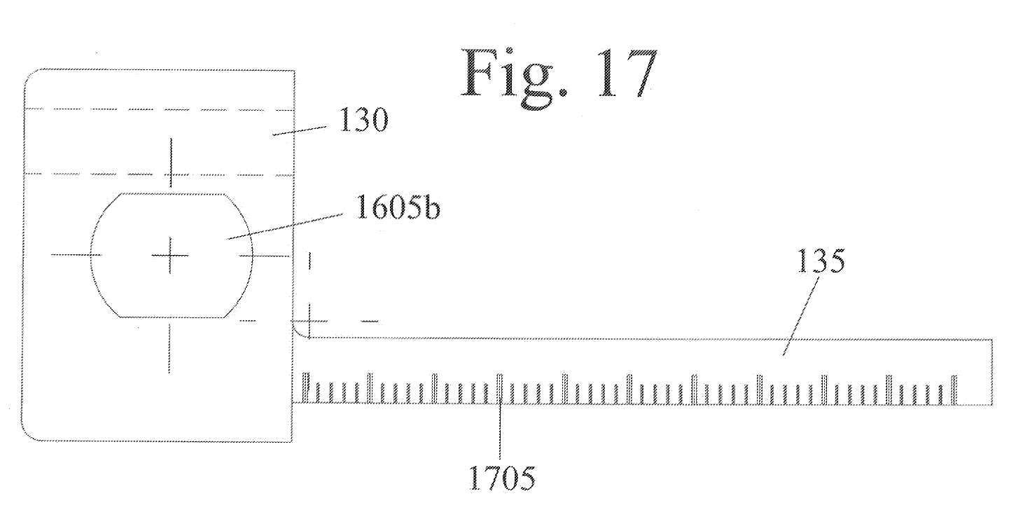

In one embodiment, the mounting system includes a size indicator that provides a measurement indication of the distance between the bone screws 125 and, therefore, the size of the cross-member 205 that would be required to link the bone screws. For example, with reference to FIG. 17, at least one of the external cross-members 135 include hatch marks 1705 along the length of the external cross-member. The hatch marks 1705 provide a visual indication of the distance between the bone screws 125 and of the cross-member size for linking the bone screws. The hatch marks may provide an actual measure of the implanted cross-member size in a recognized physical unit or simply give an arbitrary designation by which the internal cross-members 205 are labeled.

The illustrated mounting system is an exemplary embodiment and it should be appreciated that other arrangements are possible and can be equally adapted to receive and guide the placement instrument 102. Since the mounting system functions to define the position of the distal end of each bone fastener and to make them co-planar with the insertion ports (proximal openings) of support members 120 (and, therefore, with instrument 102), alternative mounting systems could be easily configured to achieve this function. For example, after the support members and bone fasteners are inserted, the insertion ports of the support members may be reversibly joined so to produce a three-point triangular arrangement. The three points would include the poly-axial proximal segment of one bone fastener, the poly-axial proximal segment of the second bone fastener, and the joined proximal segments (insertion port ends) of the support members. This arrangement would align the bone fasteners and allow cross-member insertion using instrument 102. Alternatively, the mounting system may be configured to position the support members in a co-planer but non-parallel configuration where the two proximal segments (insertion port ends) are not joined together. This latter configuration would also align the bone fasteners and allow cross-member insertion using instrument 102.

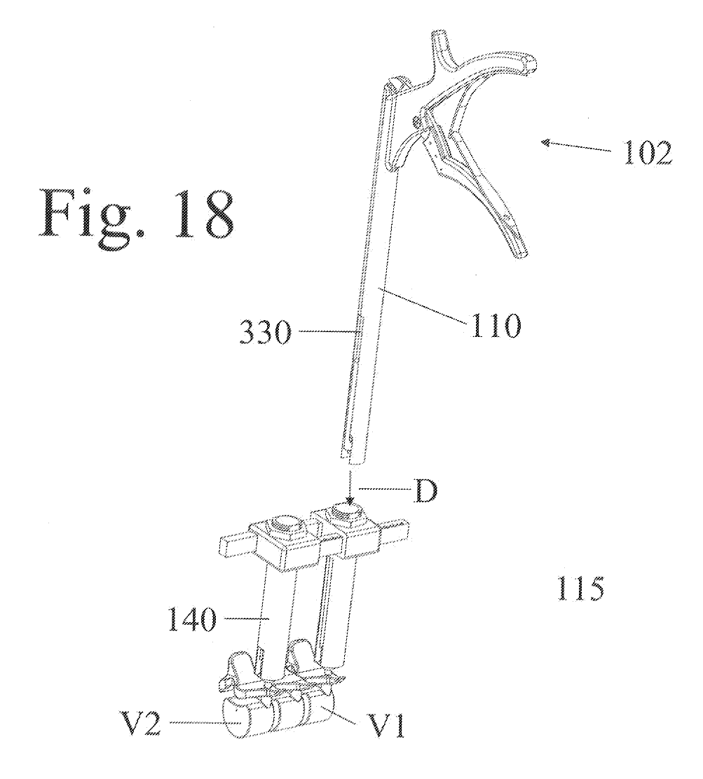

FIG. 18 shows a perspective view of the assembled mounting system 115 attached to the vertebral bodies V1 and V2. For clarity of illustration, other anatomical structures, such as skin tissue and additional skeletal segments, are not shown in FIG. 18 or the other Figures herein. With the mounting system 115 attached to the vertebral bodies, the instrument 102 is positioned for coupling to the mounting system 115. The instrument 102 is coupled to the mounting system by inserting the housing 110 through an insertion port into the internal shaft of the support member 120a, as represented by the arrow D in FIG. 18. During coupling, the instrument 102 is oriented such that the slot 330 on the housing 110 aligns with the slot 140 on the support member 120. Correct orientation of the placement device within the support member is assured by the interaction of protrusion 1110 of housing 110 (FIG. 4) with a complimentary indentation on the inner aspect of each support member 120.

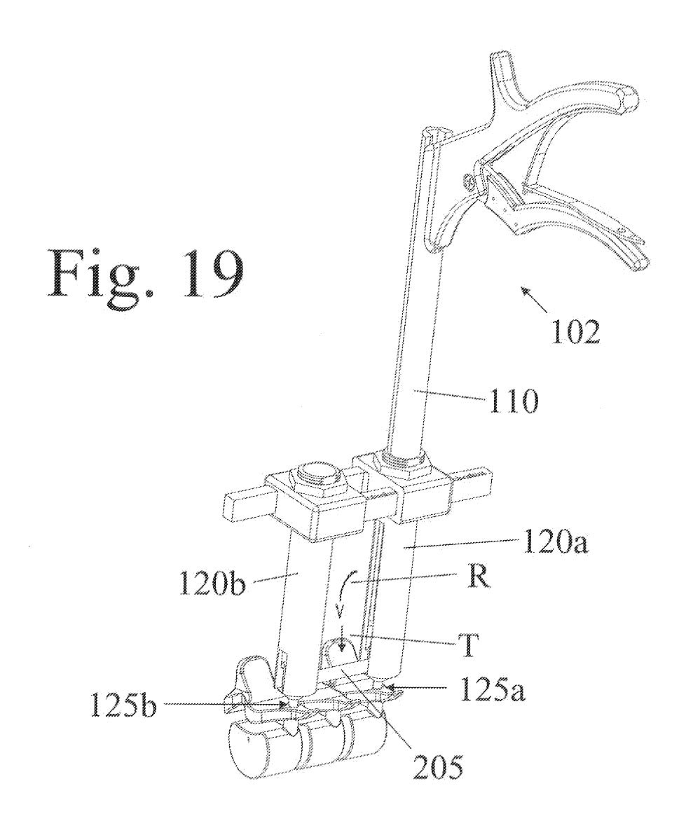

With reference now to FIG. 19, the instrument 102 is pushed distally into the support member 120a until the instrument is seated in the support member 120. The instrument 102 is now coupled to the mounting system. The instrument 102 is then actuated, such as by pulling the trigger handle 320 toward the handle 310. It should be appreciated that the instrument 102 can be actuated in manners other than pulling a trigger handle. For example, in one embodiment, the internal mechanism can be removably coupled to a driving instrument, such as drill, that is activated to impart a driving or other type of actuation force that actuates the internal mechanism. All or a portion of the internal mechanism can also be replaced or complimented with electro-mechanical components.

Actuation of the instrument 102 causes the internal deployment mechanism to deploy the cross-member 205 out of the housing 110. As discussed above, during deployment the cross-member 205 rotates or pivots out of the housing 110 (as represented by the arrow R in FIG. 19) and then translates distally toward the receiver member of the screw 125 (as represented by the arrow T in FIG. 19). The alignment of the slot 330 in the instrument housing 110 and the slot 140 in the support member 120 permits the cross-member 205 to be deployed out of the housing 110.

With the cross-member 205 positioned as shown in FIG. 19 a locking nut is placed into the support member 120b and used to lock the cross-member 205 within the receiver member of the second bone screw 125b. The hand operated instrument is then removed from the first support member 120a and a locking nut is used to lock the other end of the cross-member 205 within the first bone screw 125a. The support members 110 and platforms 130 are then removed leaving the bones fixed together.

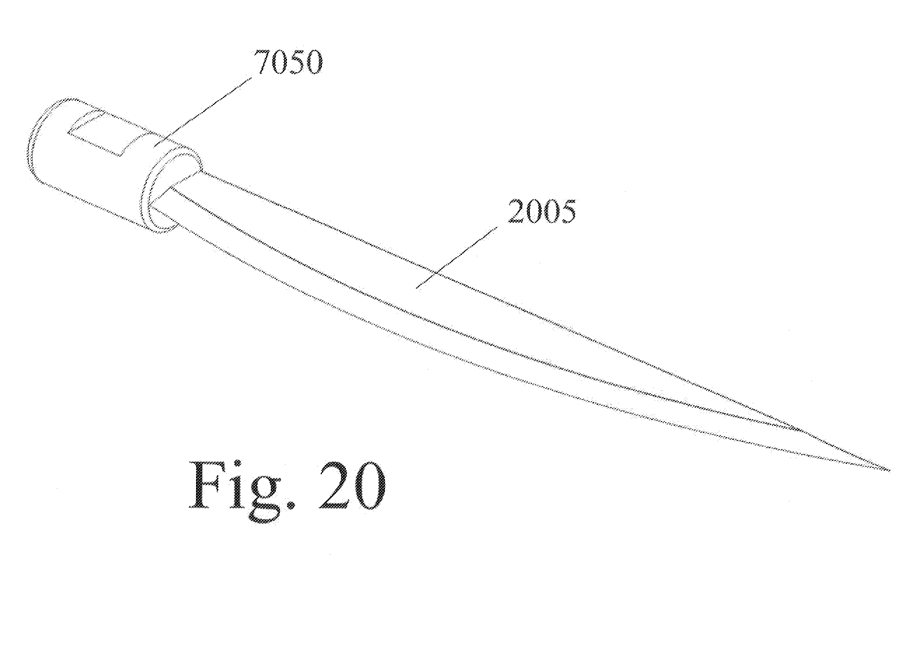

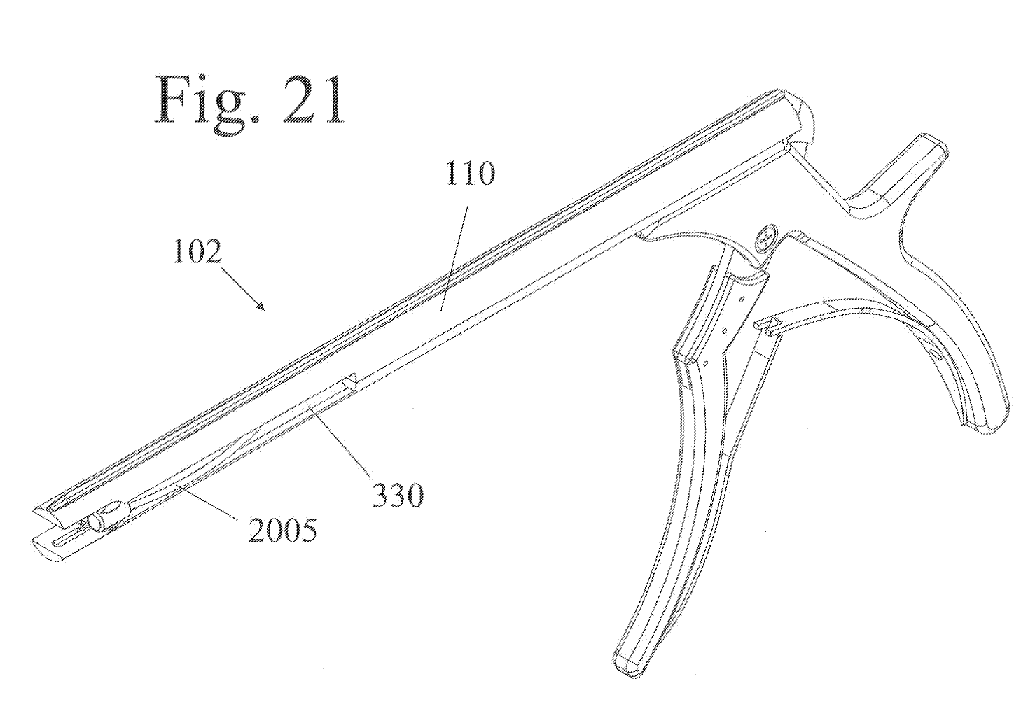

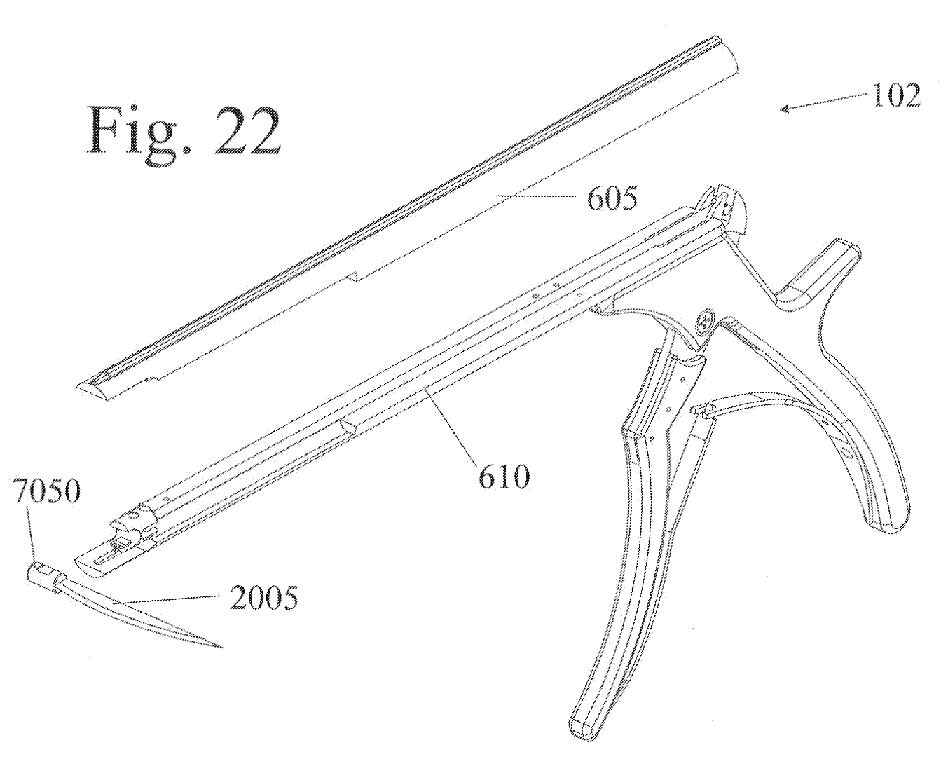

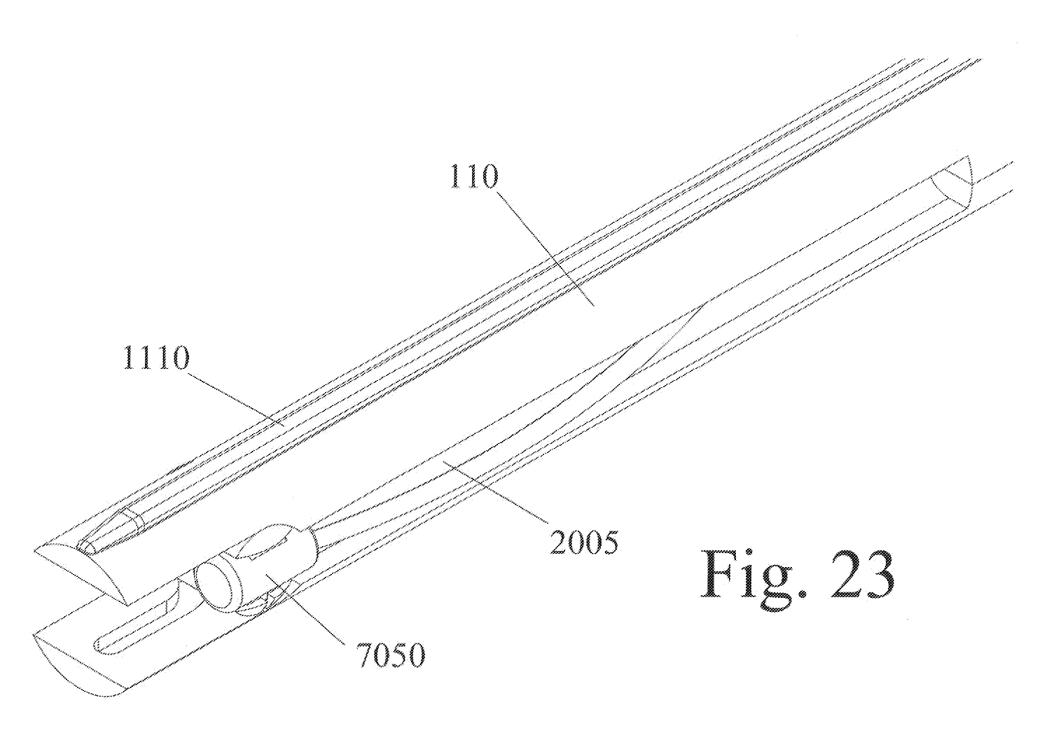

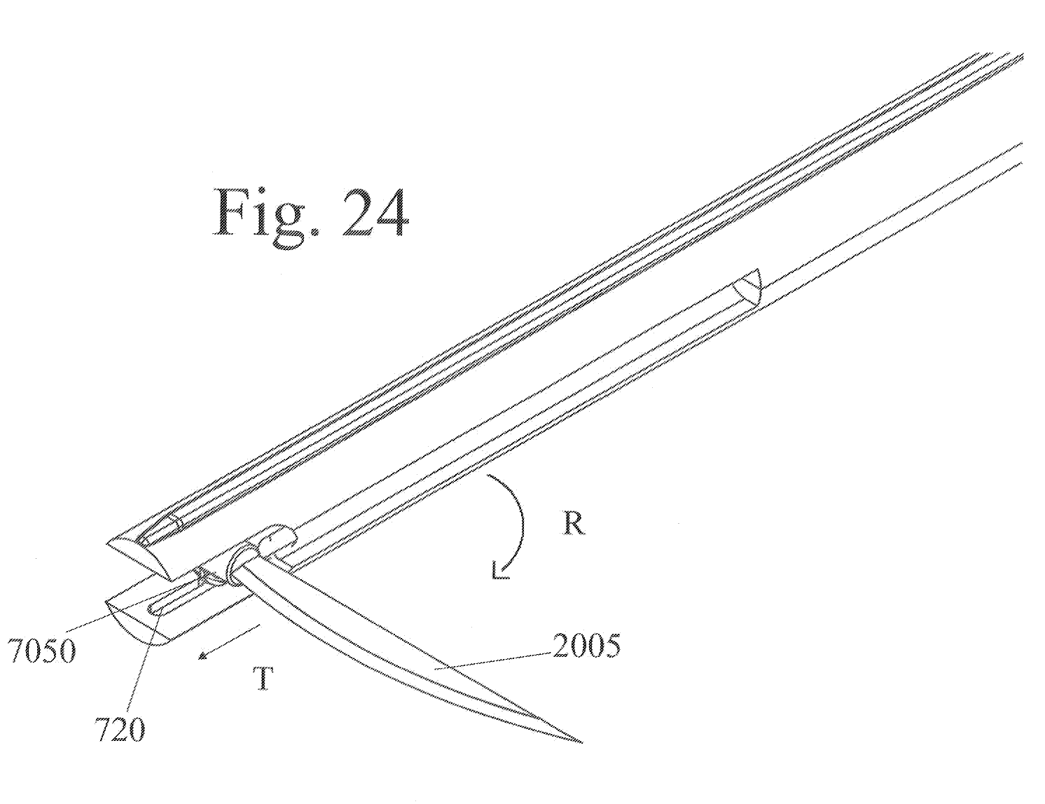

FIG. 20 shows another embodiment of a device that can be used with the instrument 102, In this embodiment, the holder 705 is attached to a knife member having a sharpened region 2005 (such as a sharpened edge that can cut through tissue) and a attachment segment 7050. FIG. 21 shows the instrument 102 with the knife member positioned in a pre-deployed state. FIG. 22 shows the instrument 102 in an exploded state. In the predeployed state, the knife 2005 is positioned within slot 330 of housing 110, as shown in the enlarged view of the distal region of the instrument 102 in FIG. 23. Upon initial actuation of the instrument 102, the knife 2005 rotates out of the housing 110 to the position shown in FIG. 24. The rotation is represented by the arrow R in FIG. 24. Upon further actuation of the instrument 102, the knife 2005 translates in the distal direction T. As discussed above with respect to the previous embodiment, the knife travels along a pathway that is dictated by the guideway 720 that interfaces with the holder 705.

In application, as assembled mounting system 115 is attached to the vertebral bodies V1 and V2 as illustrated above. The knife adapter of FIG. 20 is attached onto the placement instrument 102 as shown in FIG. 21 and then instrument 102 is inserted into the internal shaft of one support member 120 of the mounting system 115. Actuation of the handle of instrument 102 causes the knife to deploy and cut through the slot tissues between the two support members 120 in order to prepare a pathway for the placement of the cross-member 205. After returning to the pre-deployed configuration, instrument 102 is withdrawn and the knife adapter is removed. A cross-member 205 of appropriate length is coupled to the instrument 102 and delivered to the operative site as discussed above and illustrated in FIGS. 18 and 19. Use of the knife adapter is an optional but preferred step that serves to cut the soft tissue and prepare the placement site for cross-member deployment. In should be appreciated that the knife adapter may be a separate member that attaches onto instrument 102, as shown, or it may be alternatively incorporated with holder 705 into a single, unitary member.

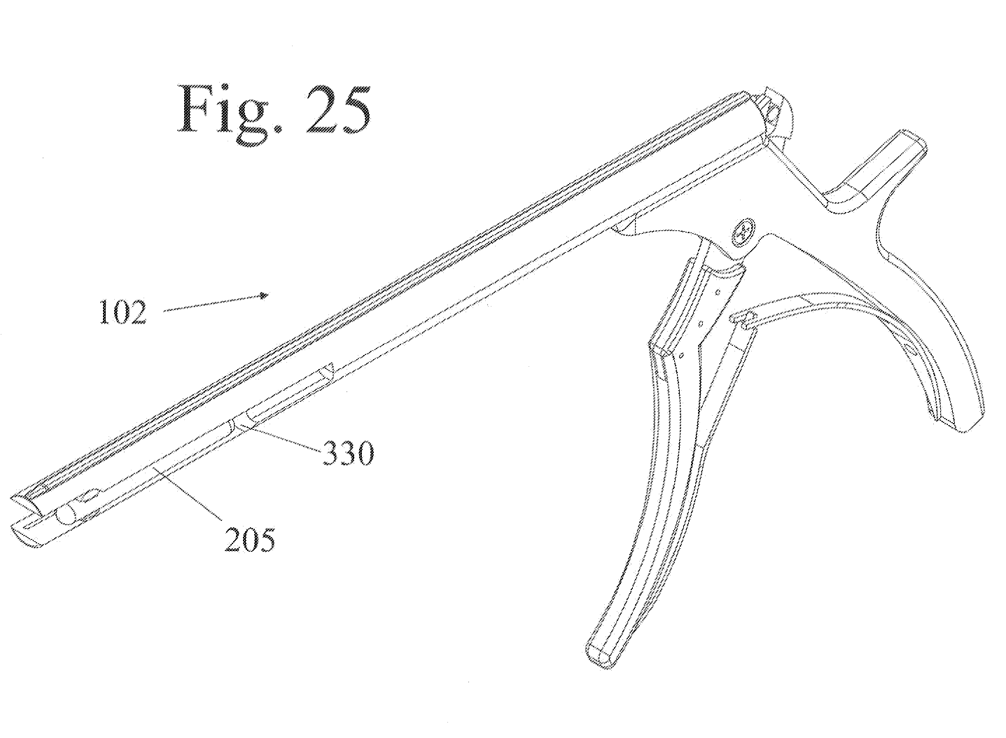

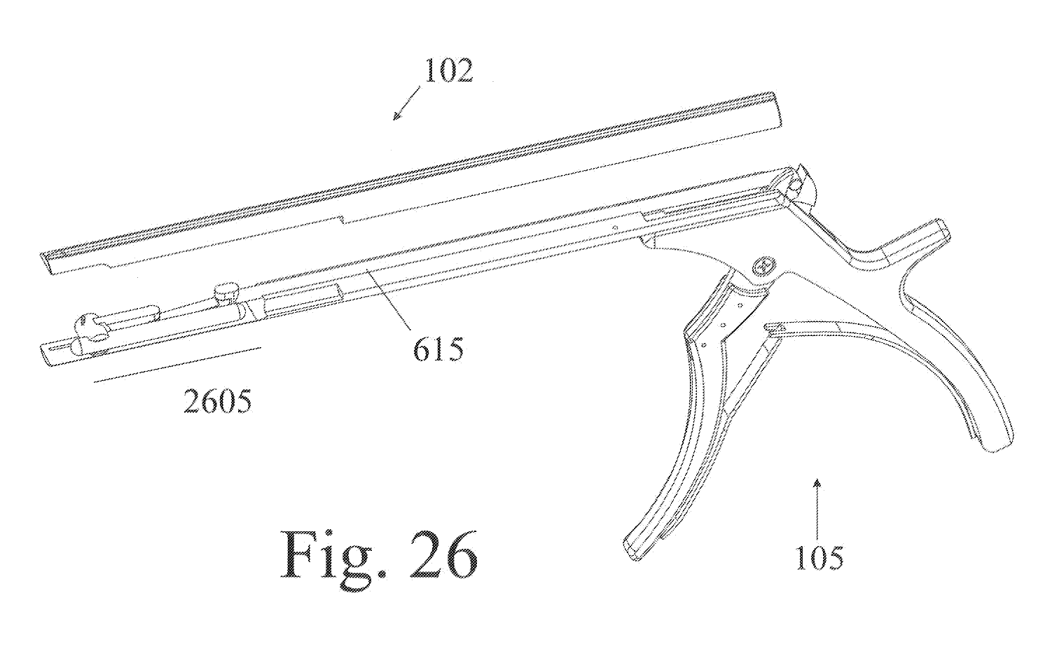

FIG. 25 shows another embodiment of the instrument 102 that includes an alternate embodiment of the internal actuation mechanism. FIG. 26 shows an exploded view of the instrument of FIG. 25. The alternate embodiment of the actuation mechanism is described in the context of deploying a cross-member 205 although it could be used to deploy other devices, such as the knife described above. In this embodiment, the distal end of the deployment member 615 is attached to a mechanism 2605 that rotates and translates the cross-member 205 upon actuation of the handle 105.

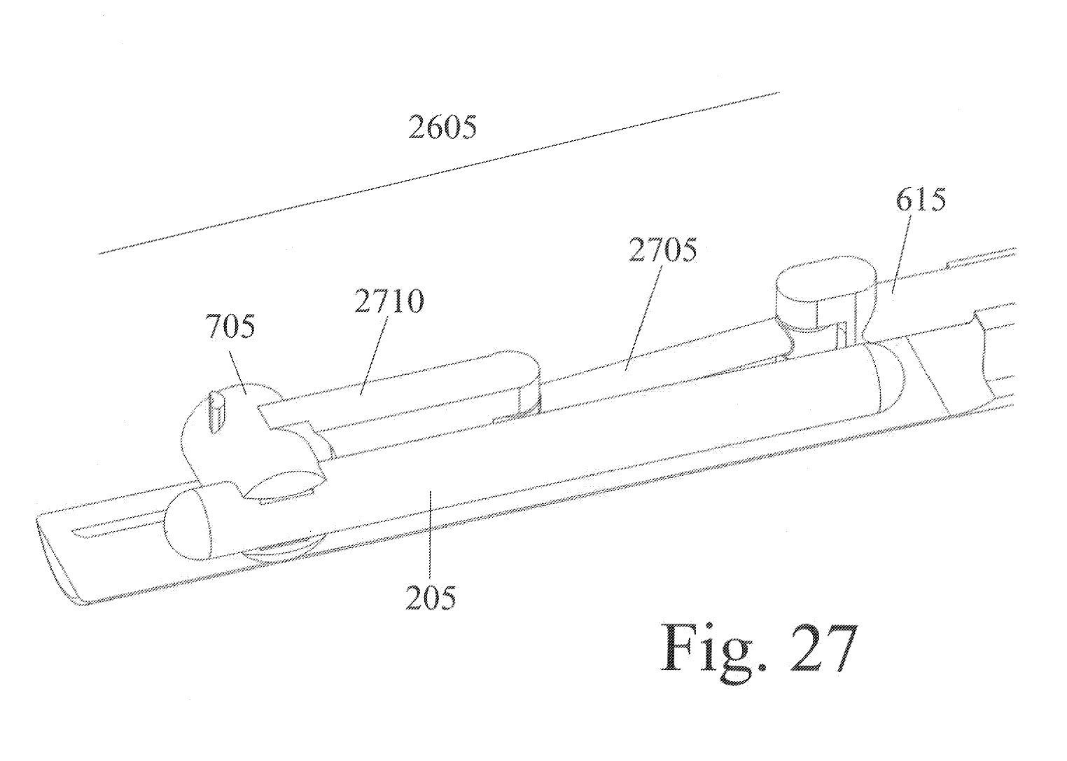

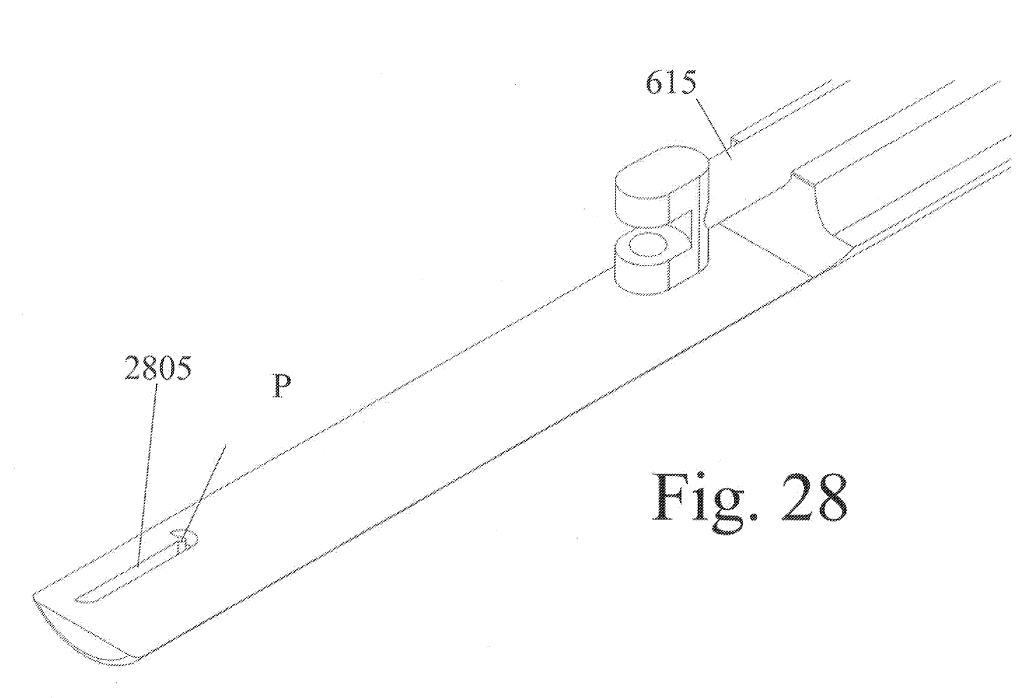

FIG. 27 shows an enlarged view of the mechanism 2605. The mechanism 2605 includes a first linkage 2705 that is rotatably attached to the distal end of the deployment member 615. A second linkage 2710 is rotatably attached to the first linkage 2705 and non-rotatably attached to the holder 705, which holds the cross-member 205. FIG. 28 shows the distal region of the instrument with a portion of the mechanism 2605 removed. A guideway 2805 is positioned to interface with a protrusion on the holder 705 and guide the holder along a predetermined pathway during actuation of the instrument.

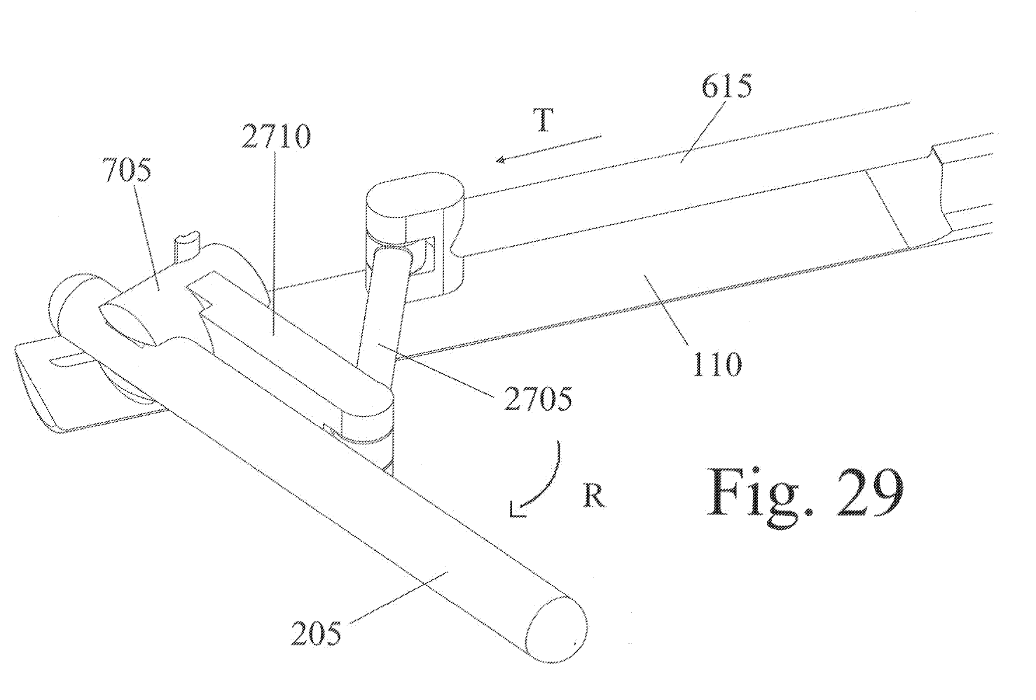

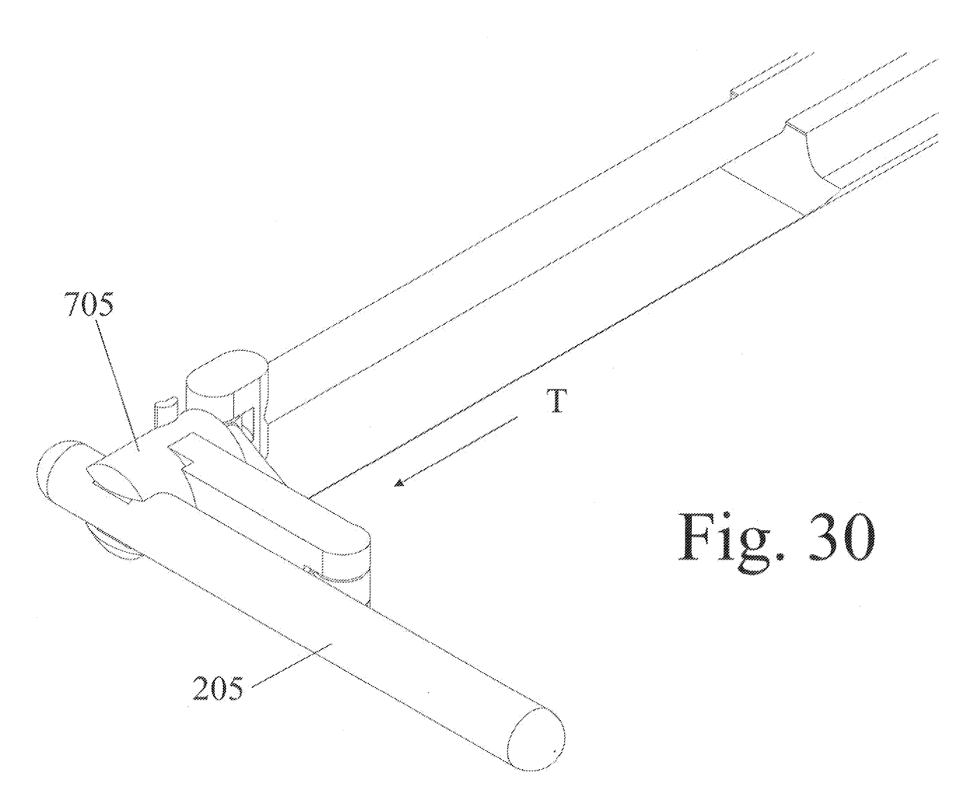

The actuation procedure is described in more detail with reference to FIGS. 27, 29 and 30, which show the action of the mechanism 2605 during actuation of the instrument. As mentioned, in the pre-deployed state, the cross-member 205 is positioned inside the housing 110 as shown in FIG. 27. For clarity of illustration, the entire housing is not shown in FIGS. 27-30 although, in actual use, the mechanism is contained within the housing as shown in FIG. 25. Upon actuation of the instrument 102 (such as by actuating the handle 105), the deployment member 615 moves in the distal direction T. This causes the linkage 2705 and 2710 to swing outward such that the cross-member 205 rotates or pivots outward along direction R. During such rotation, the holder 705 rotates about a pivot point P along the guideway 2805 (FIG. 28). The cross-member 205 rotates to a position wherein it is perpendicular to the long axis of the housing 110 and then the cross-member translates along the distal direction T until It reaches the deployment position shown in FIG. 30.

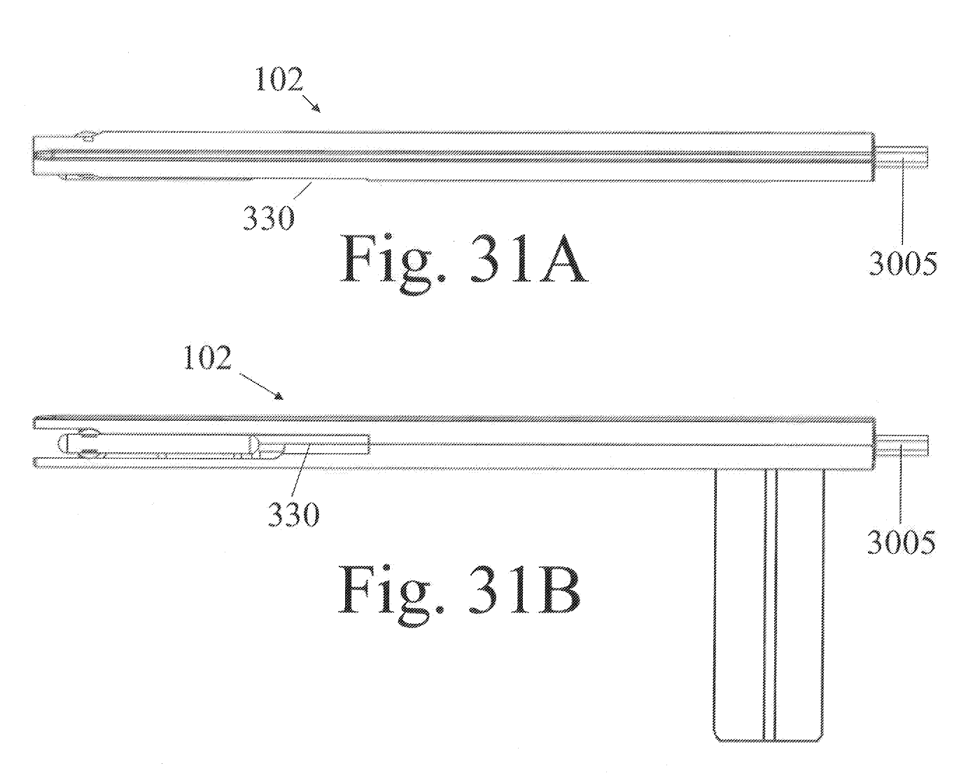

The embodiment of the instrument described above can be actuated by squeezing the trigger handle 105 or by another type of hand actuation. Any embodiment of the instrument 102 can be alternatively actuated by using a separate rotatory device, such as a drill. FIGS. 31A, and 31B show top and side views of an instrument 102 that is actuated by coupling to a hand-powered or motorized drill. In this regard, the instrument 102 includes an attachment 3005 that is adapted to be coupled to a rotatory driver and attachment 3005 can have any of a variety of shapes and sizes that are adapted for coupling to a drill. Upon coupling, the drill is activated to cause the attachment 3005 to rotate and actuate the internal mechanism.

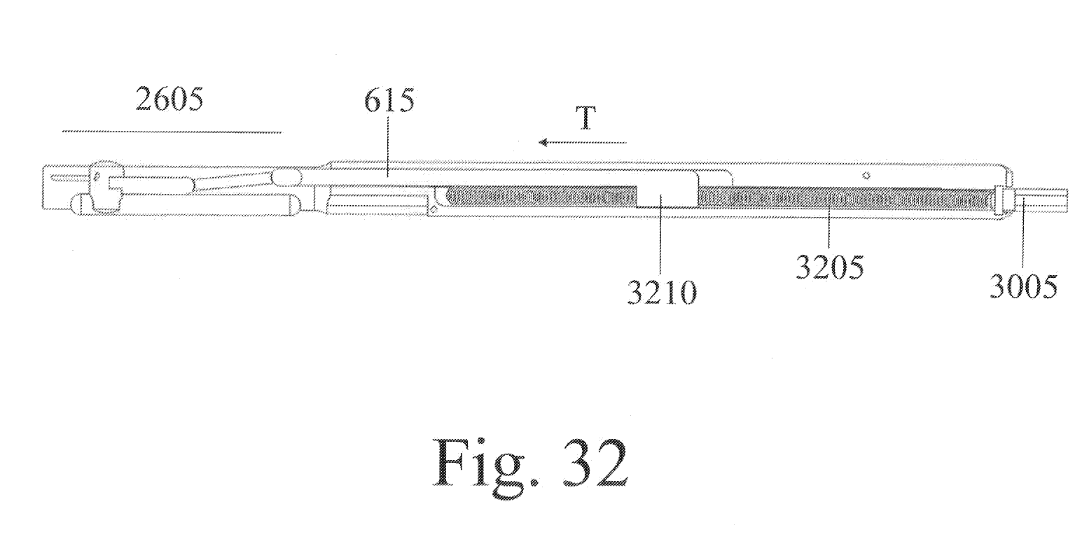

FIG. 32 shows an exemplary embodiment of an internal mechanism. The attachment 3005 is coupled to a threaded lead screw 3205, which interacts with the deployment member 615 via an internally threaded segment 3210. When activated, the drill rotates attachment 3005 and threaded lead screw 3205, which causes the deployment member 615 to move along direction T because of the interaction between the externally threaded lead screw 3205 and the internally threaded segment 3210. With movement of member 615, mechanism 2605 deploys the cross-member 205 in the manner described above with reference to FIGS. 27-29. Rotation of member 3005 in one direction will produce deployment of the cross-member 205 while rotation in the opposite direction will cause member 205 to return into the confines of opening 330 of the device (un-deployed configuration of FIG. 30).

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable material and the like. Any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, a surface of any of the implants may be made with a porous ingrowth surface (such as titanium wire mesh, plasma-sprayed titanium, tantalum porous, CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Lastly, any assembly or its components can also be entirely or partially made of a shape memory material or other deformable material.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed is:

1. An instrument or placing a cross-member between a pair of bone screws, comprising:
an actuator;
a housing coupled to the actuator, the housing containing the cross-member in a pre-deployed state wherein the cross-member is at least partially enclosed within the housing; and
a mechanism coupling the actuator to the cross-member, wherein actuation of the actuator causes the mechanism to rotate the cross-member out of the housing and to further translate the cross-member in a direction toward engagement with a bone screw.

2. An instrument as in claim 1, wherein the actuator can be actuated using a single hand.

3. An instrument as in claim 1, wherein the actuator includes handle having a trigger that is pulled to actuate the mechanism.

4. An instrument as in claim 1, wherein the housing comprises an elongate, tubular structure having an internal cavity that contains the cross-member.

5. An instrument as in claim 1, wherein the actuator can be coupled to a drill for actuating the mechanism.

6. A system for placing a cross-member between first and second bone screws, comprising:
an instrument comprising:
(a) an actuator;
(b) a housing coupled to the actuators, the housing containing the cross-member in a pre-deployed state wherein the cross-member is at least partially enclosed within the housing; and
(c) a mechanism coupling the actuator to the cross-member, wherein actuation of the actuator causes the mechanism to rotate the cross-member out of the housing and to further translate the cross-member in a direction toward engagement with a bone screw; and
a mounting system including at least one mounting member that aligns the instrument within a plane that includes the first and second bone screws.

7. A system as in claim 6, wherein the mounting system includes a first elongate member that attaches to the first bone screw and a second elongate member that attaches to the second bone screw, wherein the first and second elongate members include internal shafts that are sized to receive at least a portion of the instrument.

8. A system as in claim 6, wherein the first and second elongate members are positioned within the plane.

9. A system as in claim 6, wherein the first and second elongate members are parallel to one another.

10. A system as in claim 6, wherein the mounting system further includes a measurement device for measuring the distance between the first and second bone screws.

11. A system as in claim 6, wherein the housing of the instrument includes an elongate slot for deployment of the cross-member and wherein the first and second elongate members each include an elongate slot that can be aligned with the elongate slot in the housing during insertion of the housing into the elongate members.

12. An instrument for positioning a device relative to a pair of bone screws, comprising:
an actuator;
a housing coupled to the actuator, the housing containing the device in a pre-deployed state wherein the device is at least partially enclosed within the housing; and
a mechanism coupling the actuator to the device, wherein actuation of the actuator causes the mechanism cause the device to follow a curvilinear path out of the housing and to further translate the device in a direction toward the bone screws.

13. An instrument as in claim 12, wherein the device is a knife.

14. An instrument as in claim 13, wherein the device is a cross-member adapted to link the pair of bone screws.

15. An instrument as in claim 12, wherein the instrument is actuated by imparting a rotational force to the instrument.

16. An instrument as in claim 12, wherein the actuator can be coupled to a drill for actuating the mechanism.

17. An instrument for positioning a device relative to a pair of bone screws, comprising:
an actuator;
a housing coupled to the actuator, the housing containing the device in a pre-deployed state wherein the device is at least partially enclosed within the housing; and
a mechanism coupling the actuator to the device, wherein actuation of the actuator causes the mechanism to rotate the device out of the housing in a direction toward the bone screws.

18. An instrument for positioning a device relative to a pair of bone screws, comprising:
an actuator;
a housing coupled to the actuator, the housing containing the device in a pre-deployed state wherein the device is at least partially enclosed within the housing; and
a mechanism coupling the actuator to the device, wherein actuation of the actuator causes the mechanism to translate the device out of the housing in a direction toward the bone screws.

19. A method of placing an implant relative to a pair of bone screws, comprising:
providing an instrument having the implant contained at least partially within the instrument;
actuating the instrument so that the instrument deploys the implant by causing the implant to rotate out of the instrument and translate toward a deployment position relative to the bone screws; and
attaching the implant to the bone screws.

20. A method as in claim 19, further comprising mounting the instrument on a mounting system that orients the instrument relative to the bone screws.

21. A method as in claim 20, wherein the mounting system positions the instrument in a plane that includes the bone screws.

22. A method as in claim 19, further actuating the instrument to deploy a knife by causing the knife to rotate out of the instrument and translate toward the bone screws such that the knife cuts through tissue to form a passageway to the bone screws.

23. A method as in claim 19, wherein the same instrument is used to deploy the implant and to deploy the knife.

24. A method as in claim 19, wherein separate instruments are used to deploy the implant and to deploy the knife.

25. A device adapted to be coupled to a spinal implant placement instrument;
a knife portion;
a coupling portion attached to the knife portion, the coupling portion adapted to be coupled to an instrument such that the knife portion is at least partially contained in the instrument, wherein the instrument is adapted to rotate and translate the knife portion out of the instrument.
